(19) United States
(12) Patent Application Publication
(10) Pub. No.: US 2004/0048375 A1
(43) Pub. Date: Mar. 11, 2004

(54) Application of pluripotential stem cells for body organ tissue repair and venous capillary expansion

(75) Inventor: Eckhard Alt, Ottobrunn (DE)

Correspondence Address: DONALD R. GREENE, P.O. BOX 12995, SCOTTSDALE, AZ, 85267, US

(73) Assignee: SciCoTec GmbH, Munich (DE)

(21) Appl. No.: 10/387,230

(22) Filed: Mar. 11, 2003

Related U.S. Application Data
(63) Continuation-in-part of application No. 09/968,739, filed on Sep. 30, 2001.

(51) Int. Cl. C12N 5/08; C12N 5/00

(52) U.S. Cl. 435/373; 435/366

(57) ABSTRACT

A method of repairing tissue of an organ in a patient's body includes co-culturing pluripotential stem cells obtained from the patient's body with cells obtained from a site other than the patient's body (i.e., non body cells) and having specific functions of the tissue to be repaired for mimicking by the stem cells and creating a specific microenvironment, maintaining the culture for a period of time sufficient for modification of the stem cells by acquisition of the specific functions of the non body cells; segregating the modified stem cells from the non body cells, and implanting only the modified stem cells to and at a site of the tissue to be repaired.

### CROSS-REFERENCE TO RELATED APPLICATION

The present application is a continuation-in-part of co-pending application Ser. No. 09/968,739 filed Sep. 30, 2001, titled “Transluminal Application of Myogenic Cells for Body Organ Tissue Repair” (“the '739 application”) of the same inventor.

### BACKGROUND OF THE INVENTION

The present invention relates generally to application of pluripotential, e.g., mesenchymal, stem cells for tissue repair, such as myocardial repair, and more particularly to transformation of pluripotential stem cells to perform highly specific functions for replacement or repair of dead or failing tissue in body organs such as the heart, brain, liver, kidney or pancreas. It is a principal aim of the invention to provide novel methods of repair or replacement of damaged or necrose tissue.

In principle, the human body has three types of cells. One type constitutes cells that continuously undergo replication and reproduction, such as dermal cells and epithelial cells of the intestine, for example. These cells, which have a life as short as ten days, are replaced by the same cell type that is replicating continuously. A second type of cell is differentiated in the adult state, but has the potential to undergo replication and the ability to reenter the cell cycle under certain conditions, an example being liver cells. The liver has the capacity to regrow and repair itself even if a tumor is excised and a major portion of the liver is removed. The third cell type comprises those cells that stop dividing after they have reached their adult stage, such as neuro cells and myocardial cells.

For the latter type or group of cells, the number of cells in the body is determined shortly after birth. For example, myocardial cells stop dividing at about day ten after delivery, and for the rest of its life the human body has a fixed number of myocardial cells. Changes in myocardial function occur not by division and new cell growth, but only as a result of hypertrophy of the cells.

Although the absence of cell division in myocardial cells is beneficial to prevent the occurrence of tumors—which practically never occur in the heart—it is detrimental with regard to local repair capacities. During the individual's lifetime, myocardial cells are subjected to various causes of damage, that irreversibly lead to cell necrosis or apoptosis.

The primary reason for cell death in the myocardium is ischemic heart disease—in which the blood supply to the constantly beating heart is compromised through either arteriosclerotic build-up or acute occlusion of a vessel following a thrombus formation, generally characterized as myocardial infarction (MI). The ischemic tolerance of myocardial cells following the shut-off of the blood supply is in a range of three to six hours. After this time the overwhelming majority of cells undergo cell death and are replaced by scar tissue.

Myocardial ischemia or infarction leads to irreversible loss of functional cardiac tissue with possible deterioration of pump function and death of the individual. It remains the leading cause of death in civilized countries. Occlusion of a coronary vessel leads to interruption of the blood supply of the dependent capillary system. After some 3 to 6 hours without nutrition and oxygen, cardiomyocytes die and undergo necrosis. An inflammation of the surrounding tissue occurs with invasion of inflammatory cells and phagocytosis of cell debris. A fibrotic scarring occurs, and the former contribution of this part of the heart to the contractile force is lost. The only way for the cardiac muscle to compensate for this kind of tissue loss is hypertrophy of the remaining cardiomyocytes (accumulation of cellular protein and contractile elements inside the cell), since the ability to replace dead heart tissue by means of hyperplasia (cell division of cardiomyocytes with formation of new cells) is lost shortly after the birth of mammals.

Other means of myocardial cell alteration are the so-called cardiomyopathies, which represent various different influences of damage to myocardial cells. Endocrine, metabolic (alcohol) or infectious (virus myocarditis) agents lead to cell death, with a consequently reduced myocardial function. The group of patients that suffer myocardial damage following cytostatic treatment for cancers such as breast or gastrointestinal or bone marrow cancers is increasing as well, attributable to cell necrosis and apoptosis from the cytostatic agents.

Heretofore, the only means for repair has been to provide an optimal perfusion through the coronary arteries using either interventional cardiology—such as PTCA (percutaneous transluminal coronary angioplasty) balloon angioplasty or stent implantation—or surgical revascularization with bypass operation. Stunned and hibernating myocardial cells, i.e., cells that survive on a low energy level but are not contributing to the myocardial pumping function, may recover. But for those cells that are already dead, no recovery has been achieved.

The current state of interventional cardiology is one of high standard. Progress in balloon material guide wires, guiding catheters and the interventional cardiologist's experience as well as the use of concomitant medication such as inhibition of platelet function, has greatly improved the everyday practice of cardiology. Nevertheless, an acute myocardial infarction remains an event that, even with optimal treatment today, leads to a loss of from 25 to 100% of the area at risk—i.e., the myocardium dependent on blood supply via the vessel that is blocked by acute thrombus formation or chronic stenosis. A complete re-canalization by interventional means is feasible, but the ischemic tolerance of the myocardium is the limiting factor.

An article published in the New England Journal of Medicine (Schömig A. et al., “Coronary stenting plus platelet glycoprotein IIb/IIIa blockade compared with tissue plasminogen activator in acute myocardial infarction,” N Engl J Med 2000; 343:385-391), for which the applicant herein was a clinical investigator, reports on a study of the myocardial salvage following re-canalization in patients with an acute myocardial infarction. The average time until admission to the hospital in these patients was 2.5 hours and complete re-canalization was feasible after 215 minutes, roughly 3.5 hours. Nevertheless, only 57% of the myocardium at risk could be salvaged by re-canalization through interventional cardiology by means of a balloon and stent. When the group of patients was randomized to the classical thrombolytic therapy, which is the worldwide standard (with no interventional means), only 26% of the myocardium at risk could be salvaged. This means that even under optimal circumstances more than 40% of the myocardial cells are irreversibly lost.

With the knowledge that many patients arrive at a hospital at from 6 to 72 hours after the acute symptoms of complete or substantially complete vessel blockage (an acute MI), it can be assumed that the average loss of affected myocardial tissue will be found to be in a range of from 75 to 90%.

As noted above, cells can survive on a lower energy level, referred to as hibernating and stunning myocardium. As the collateral blood flow increases or re-canalization provides new blood supply they can recover their contractile function. The principle of myocardial re-perfusion, limitation of infarct size, reduction of left ventricular dysfunction and their effect on survival were described by Braunwald (Braunwald E. et al., “Myocardial reperfusion, limitation of infarct size, reduction of left ventricular dysfunction, and improved survival: should the paradigm be expanded?,” Circulation 1989; 79:441-4).

Annually, about five million Americans survive an acute MI. Clearly then, loss of affected myocardial tissue is a problem of major clinical importance. Currently, repair is limited to hypertrophy of the remaining myocardium, and optimal medical treatment by a reduction in pre- and after-load as well as the optimal treatment of the ischemic balance by β-blockers, nitrates, calcium antagonist, and ACE inhibitors.

If it were feasible to replace the dead myocardium (scar tissue) by regrowing cells, such a technique would have a profound impact on the quality of life of affected patients.

As noted earlier herein, in addition to ischemic heart disease other reasons exist for the reduction of myocardial cells that contribute to the pumping function of the heart. Among them are the cardiomyopathies, which describe a certain dysfunction of the heart. Reasons are many, such as chronic hypertension which ultimately leads to a loss in effective pumping cells, and chronic toxic noxious such as alcohol abuse or myocarditis primarily following a viral infection. Also, cell damage in conjunction with cytostatic drug treatment is becoming of greater clinical relevance.

The group of Willam C. Claycomb et al. has been engaged in research on the behavior and the development of myocytes since the early 1970's. In their initial report (Goldstein M. A. et al., “DNA synthesis and mitosis in well-differentiated mammalian cardiocytes,” Science 1974; 183:212-3), they described the incorporation of 3H-Thymidin into the nuclei of heart cells of two days old rats, which indicates that neonatal cardiac cells still undergo synthesis of DNA and divide despite the presence of contractile proteins. This phenomenon of cell division ceases at day 17 of the postnatal development. After that time no further division of cardiac cells occurs—in rats or in humans.

The interest in mammalian cardiomyocytes has led to the development of cultures of adult cardiac muscle cells (Claycomb W. C. et al., “Culture of the terminally differentiated adult cardiac muscle cell: A light and scanning electron microscope study,” Dev Biol 1980;80:466-482), and ultimately to the generation of a transplantable cardiac tumor-derived transgenic AT1-cell.

During the 1980's intensive studies were conducted with the characterization of this atrial derived myocyte cell line, which is immortalized by the introduction of the SV40-large-T-oncogene (SV40-T). From this AT-1-cell-group, other adult cardiomyocytes have been derived. These can be passaged indefinitely in culture, can be recovered from a frozen stock, can retain a differentiated cardiomyocyte phenotype, and maintain their contractile activity. They are described as HL-1-cells. The reader is referred, for example, to Delcarpio J. B. et al., “Morphological characterization of cardiomyocytes isolated from a trans-plantable cardiac tumor derived from transgenic mouse atria (AT-1 cells),” Circ Res 1991; 69(6):1591-1600; Lanson Jr. N. A. et al., “Gene expression and atrial natriuretic factor processing and secretion in cultured AT-1 cardiac myocytes,” Circulation 1992; 85(5):1835-1841; Kline R. P. et al., “Spontaneous activity in transgenic mouse heart: Comparison of primary atrial tumor with cultured AT-1 atrial myocytes,” J Cardiovasc Electrophysiol 1993; 4(6):642-660; Borisov A. B. et al., “Proliferative potential and differentiated characteristics of cultured cardiac muscle cells expressing the SV 40 T oncogene,” Card Growth Reg 1995; 752:80-91; and Claycomb W. C. et al., “HL-1 cells: A cardiac muscle cell line that contracts and retains phenotypic characteristics of the adult cardiomyocyte,” Proc Natl Acad Sci USA 1998; 95:2979-84.

Finally, the cardiomyocyte transplantation in a porcine myocardial infarction model has been studied intensively in collaboration with the research group of Frank Smart (Watanabe E. et al., “Cardiomyocyte transplantation in a porcine myocardial infarction model,” Cell Transplant 1998; 7(3):239-246). In conjunction with the AT-1 cardiomyocytes, human fetal cardiomyocytes were injected into the adult pig heart infarction area.

In summary, these cells showed local growth and survived in the infarction border zone, but could not be found in the core scar tissue of the myocardial infarction. The majority of the implanted cells were replaced with inflammatory cells, suggesting that the immuno-suppressant regimen that was concomitantly applied was not sufficient for the grafted cells to survive in the host myocardium. Other factors that may have influenced the result that the transplanted cells were not detected, could possibly be linked to the fact that the cells were grafted 45 days after inducing the infarction.

It is known that the inflammatory stimuli for cell growth are significantly reduced in the first two to three weeks following an MI. Also, that transforming-growth-factor-b (TGF-b), fibroblast-growth-factor-2 (FGF-2), platelet-derived-growth-factor (PDGF) and other cytokines, like the interleucin-family, tumor-necrosis-factor-a (TNF-a) and interferon-g are strong stimulators of cell proliferation and cell growth. The adjunct therapy with immuno-suppression has further reduced these stimuli for cell growth.

Another major factor for the failure of detection of grafted cells in the myocardial scar may be the selection of the infarction model. An artery is occluded and the blood supply has not recovered before grafting. There is no reason to assume that the grafted cells could survive in an ischemic area and grow, better than the myocytes.

Therefore, other groups have tried to induce a myocardial angiogenesis by gene-therapy. This was either performed by the administration by fibroblast growth factor II in the presence or absence of heparin (see Watanabe E. et al., “Effect of basic fibroblast growth factor on angiogenesis in the infarcted porcine heart,” Basic Res Cardiol 1998; 93:30-7) or by application of vascular endothelial growth factor (VEGF), a potent mitogen for endothelial cells. VEGF stimulates capillary formation and increases vascular permeability (Lee J. S. et al., “Gene therapy for therapeutic myocardial angiogenesis: A promising synthesis of two emerging technologies,” Nat Med 1998; 4(6):739-42). Still other groups have tried to increase the collateral capillary blood flow by human bone marrow derived angioblasts and have shown an improvement in acute myocardial infarction in rats treated with injections of colony-stimulating-factor-G (CSF-G) mobilized adult human CD-34 cells (Kocher A. A. et al., “Neovascularization of ischemic myocardium by human bone-marrow-derived angioblasts prevents cardiomyocyte apoptosis, reduces re-modeling and improves cardiac function,” Nat Med 2001; 7(4):430-6).

While these approaches certainly have some research merit, their clinical relevance for the majority of patients is not as important because of the immunogenic and neoplastic risks associated with genetically modified cells.

Other attempts to transplant preformed patches also necessitate the growth of the grafted cells in a patch formation and a surgical operation in a patient, which requires opening the thoracic cage.

Considering the complications, the cost and the risk associated with these time consuming procedures, it becomes clear that they offer only limited likelihood for widespread routine application.

Other groups have tried to make use of the precursor cells that are found in the peripheral muscle. Unlike the heart, there is a certain degree of repair in peripheral skeletal muscles, since the peripheral skeletal muscle contains progenitor cells, which have the capability to divide and replace the peripheral muscle. By isolating those cells from a probe of a thigh muscle, the progenitor cells of skeletal muscle have been separated, cultured and re-injected in an animal model (Taylor D. A. et al., “Regenerating functional myocardium: Improved performance after skeletal myoblast transplantation,” Nat Med 1998; 4(8):929-33; Scorsin M. et al., “Comparison of the effects of fetal cardiomyocyte and skeletal myoblast transplantation on postinfarction left ventricular function,” J Thorac Cardiovasc Surg 2000; 119:1169-75), and more recently in some patients also.

The application of these cultured cells also been attempted by injection with small needles following an opening of the subject's chest and the pericardial sac. While in the model of kryo-infarction, in which only the myocardial cells die but the blood supply through the vascular system is not limited, the injection of autologous skeletal myoblasts improves the myocardial function. The results indicated, however, that the engrafted cells retain skeletal muscle characteristic, which means they cannot contract at the constant fast rate imposed by the surrounding cardiac tissue. In addition, no electrical connection exists between the graft cells and the host tissue, and it is assumed that their contribution to improved contractile performance probably resulted from the mechanical ability of the engrafted contractile tissue to respond to stretch activation by contraction.

Considering the experience with latissimus dorsi muscle grafting—a procedure called dynamic cardiomyoblasty—, the disappointing results with the possible use of skeletal muscle as a myocardial substitute indicate that the long term different muscle characteristics of skeletal muscles do not match the need of a constantly pumping myocardial cell. Therefore, the best these cells might achieve would be to improve the quality of the scar of the ischemic myocardium, but not actively contribute to a contraction of this area in the long term.

The '739 application, of which the present application is a continuation-in-part, discloses an invention directed to interventional cardiology through an intraluminal application of cells that have the capability to replace necrose tissue of a failing organ, such as myocardial tissue following an MI, to resume the myocardial function and therefore improve the pumping performance of the myocardium. The procedure is oriented toward the clinical practice of interventional cardiology, which applies the principle that only those approaches that are both (a) relatively easy to perform, with little or no risk to the patient but a potentially high benefit, and (b) highly cost effective, are likely to be routinely applied in everyday medicine.

An important aspect of the invention disclosed in the '739 application is that the cells to be used in the intraluminal or transluminal application preferably are autologous adult stem cells, which are derived from the same patient that has suffered the infarction. The cells are harvested and separated before injection, from the same individual (autologous transplantation). In a case of failing tissue of the myocardium, these cells are then injected into the coronary artery whose blockage caused the infarction.

The approach taken according to the '739 application invention recognizes that the stem cells need a certain contact time to adhere and migrate from the vascular bed into the infarcted myocardial area. In contrast to the previous approaches, in which patches or applications through needles into the infarcted area have been considered, it is hypothesized, with respect to that invention, the most effective way to deliver the cells to the infarcted area is through the vascular tree of coronary arteries, arterioles and capillaries that supply the infarcted area. An occlusion balloon of an over-the-wire type catheter is inflated at the site of the primary infarction, after the vessel has been re-canalized and the blood flow reconstituted.

Importantly, while the blood flow is still blocked, the stem cells are supplied by slow application through the balloon catheter over a relatively short period of time, on the order of 15 minutes, for example. That is, the stem cells are injected through the inner lumen of the catheter while the balloon is inflated, and therefore, no washout occurs. It is believed that this intracoronary or transcoronary application of cells during a period that perfusion is ceased is critical to enabling the cells to successfully attach to the vessel or myocardial wall. Further, this procedure is believed to overcome more actively the endothelial barrier following the increased pressure in the vascular bed, which is attributable to the fact that the retrograde blood flow is limited through the inflated balloon catheter.

Thus, the approach of the invention disclosed in the '739 application may be characterized as a method of cellular repair of failing tissue of an organ in a patient's body that comprises positioning the distal end of a catheter proximate the site of the failing tissue, and applying stem cells to the failing tissue by injection through a lumen of the catheter, while quelling local forces at the site from disrupting migration of the stem cells to the failing tissue to enhance their concentration at the site. That invention may also be stated as a method of re-canalization and reconstitution of blood flow into an ischemic organ with tissue damage, which comprises opening the ischemic organ to circulation of blood flow therethrough, and within a predetermined short time thereafter, injecting stem cells into the organ proximate the site of the damaged tissue for repair thereof. Yet another description would be of a process for repairing or replacing myocardial tissue by injecting autologous stem cells at the site of the heart where the myocardial tissue of interest is located, through a coronary artery or arterioles or capillaries thereof, and occluding the artery during cell injection downstream of the occlusion to increase the concentration of the injected stem cells and the pressure at the site to overcome an endothelial barrier between the artery and that myocardial tissue.

The principles of the '739 application invention are not limited to cellular repair of damaged or failing myocardial tissue, but may be applied in processes for repair of tissue of various organs of the body, including the brain, liver, kidney or pancreas, for example.

### SUMMARY OF THE INVENTION

It would be desirable to have a capability to replace tissue of greater specificity, i.e., having higher order functions and very specific properties—not merely a contractile muscle cell, or an endothelial cell, or a smooth muscle cell in a blood vessel—but a cell, for example, in the heart or in the brain that has even more specific functions. The heart, for example, has certain cell types possessing the capability to depolarize, i.e., an unstable phase IV potential. With calcium influx into the cell from the systole and the early diastole to the end of the diastole, the cell becomes progressively more positive and thereby reaches a threshold potential at which the fast sodium channel opens, and the cell will rapidly depolarize.

Such cells are the natural pacemaker cells, located at specific points in the heart. Those that have the fastest rate, expressed by the steepest slope of the phase IV influx and the most unstable phase IV potential, are found in the sinus node. The slope can be modified by temperature, by catecholamines, or by neurosympaticus stimulation. Normally, these cells regulate the rhythm of the heart quite consistently with a rate of about 50 beats per minute (bpm) during nighttime sleep and 200 bpm with maximum exercise, and in young patients and even some older patients at a rate of about 40 bpm at night and 150-160 bpm with exercise.

But if these cells are compromised in their function, the patient is likely to experience a very slow heart rate or even long blocks of systole, which causes syncope, fainting or limited exercise capacity. The principal therapy currently used for treating these patients is to implant an artificial pacemaker device or a defibrillator where the patient is experiencing a lack of depolarization by the cells and a missing trigger of the heart rhythm, or to ablate the tissue where the patient is suffering slow conduction areas within the heart that create reentry arrhythmias or triggered arrhythmias. The latter is performed using an ablation catheter for applying current, voltage, another form of energy, or a cooling agent, to induce a complete block.

The present invention, in one of its aspects, resides in an approach in which certain intrinsic cells derived from the patient—mesenchymal stem cells—are modified to possess the highly specific capabilities, properties and functions of, and are used to repair or replace, the compromised cells.

It is known that cells having unstable phase IV potential and immortalized can be engineered. They may be embryonic or neonatal or artificially derived cellines. For example, applicant has worked with a celline that can be grown in culture and has a property of beating fast by depolarizing spontaneously. Although such cells can be beneficial, serious concerns would be raised if those cells were to be injected into the body, such as into the heart, with respect not only to immunogenicity, but also with respect to possible late occurrence of malignancies. Since the cells are genetically modified, their genomic set may differ considerably from the patient's own cells; and further, since these cells are immortalized they have a tendency to continue growing, with the possibility that they may become malignant. Therefore, it would be undesirable to inject such cells into the body. In addition, it is likely, because these cells are foreign, they would encounter rejection by the body's immune system.

However, applicant has found that when these very specific cells, such as cells that possess the property of beating spontaneously, are co-cultured in a Petri dish with mesenchymal stem cells in a specific culture media condition, after a period of several days the latter cells assume the highly specific features of the co-cultured cells. Mesenchymal stem cells are found in young embryos, but can also readily be derived from established mesodermal layers, the bone marrow, fat, the blood or epidermis of a patient suffering from damaged or necrose tissue in need of replacement or repair.

So initially, in a method according to the present invention, the mesenchymal stem cells derived from the patient and the beating (embryonic and even animal derived depolarizing) heart cells are cultured together. The mesenchymal autologous stem cells of the patient or both cell types are marked to allow them to be identified later, and after about 10 days, merely by contact and exposure to the media and the expression of genes and release of certain proteins, the mesenchymal stem cells become beating heart cells. It remains, then, to separate the two types of cells once again, because the desire is to inject in the patient only those cells that originally came from the patient. The modified mesenchymal stem cells are then implanted at the site of the tissue to be repaired or replaced.

The present invention is not limited solely to cell pattern recognition, co-culturing and re-injection for myocardial tissue, but for tissue of other organs as well. For example, it is anticipated that the methods of the invention will also be useful for treating diseases and disorders of the brain, such as to reverse or retard the effects of Parkinson's disease.

Another aspect of the invention arises from the '739 application's teaching regarding restoration of blood flow in a blocked coronary artery of an MI patient, followed by injecting autologous adult stem cells to replace dead myocardium and repair damaged myocardium. The aspect of the present invention summarized above utilizes mesenchymal stem cells that are cultivated to mimic highly specific functional cells and are then injected for repair or replacement of damaged or dead cells. It is intended that the appropriately modified cells, when injected and implanted through the antegrade flow (i.e., through the artery), enter the capillaries where they sedate and settle, and finally convert into myocardium as their micro-environment triggers them and teaches them how to perform their new function. This is extremely important for patients who have suffered occlusion of a coronary artery, and where the artery can be re-opened and antegrade perfusion can be reinstituted.

Many cardiac patients have chronic occlusion of vessels, particularly as the patient ages. Typically, this is treated by performing bypass surgery to establish a parallel flow either through the mammary artery that has an anastomosis with the natural vessel distal from the occlusion site, or via a venous bypass graft. Such bypass solutions, however, have only a finite lifetime, typically in the range from 10 to 15 years, after which the bypass vessel also occludes. Efforts to solve this problem have, thus far, been largely futile. Attempts to re-open the natural circulation in vessels occluded for 10 or more years have had very low success rate. But, it remains important to maintain blood flow in or around these vessels. In the instance of the present invention, the modified mesenchymal stem cells are grown so as to differentiate into myocardium, but they can do so only in the presence of an active perfusion. That is, the mesenchymal cells will grow and build functional myocardial tissue provided that an adequate blood supply exists. The problem is to find a solution when the arterial vessel is occluded and cannot be re-opened.

According to a second aspect of the present invention the modified stem cells are transfected with VEGF and, for example, through a viral promoter or liposomic transfection or electroporation or magneto transfection, produce an enhanced level of VEGF, which, in culture media, has been shown to possess the capability to promote significant growth of new capillaries. But if these VEGF producing stem cells are implanted, they cannot survive in the heart unless a minimum circulation and supply of oxygen is present at the implant site. The applicant herein observed some time ago that, irrespective of how long an arterial vessel is occluded, the venous returns and their capillaries continue to function, at least to a limited extent. This means that even if an artery is occluded, the venous side of the circulation remains present. The second aspect of the invention involves a solution to the dilemma through retrograde passage. The cells are injected through the right side of the heart—through the right atrium where the coronary sinus ends—into venous vessels. By this “back door” approach to the heart, as such a procedure might be termed, the very specific region of the venous system that parallels an occluded artery may be addressed.

Each artery has a parallel vein, and if an artery is occluded the parallel vein can be entered for retrograde injection of the cells that, at least initially, will only survive in a border zone where some nutrition and flow of blood and oxygen supply persists through collaterals. In the case of retrogradely injected VEGF-producing cells, the cells or sufficient numbers of them will engraft themselves in this border zone with limited blood supply, but adequate to grow new, albeit tiny collateral vessels to aid in reducing the total ischemic area and increasing the border zone. The concept of this aspect of the present invention, then, is to inject VEGF-producing stem cells into the heart through the retrograde coronary sinus—a venous system—to induce the growth of new capillaries to provide nutrition in the form of blood flow and oxygen supply which is adequate to promote additional growth and a parallel circulation for desired cell repair or replacement. Repetitive injections to the same area will continuously reduce the size of underperfused myocardium by growing from outside to inside. The same principle of retrograde venous injection in the event of an occluded artery applies not only to the heart, but also to the leg, the intestines, the kidneys and even to the brain and other organs with arterial perfusion such as ear and eye.

### BRIEF DESCRIPTION OF THE DRAWINGS

The above and still further aims, objectives, features, aspects and attendant advantages of the present invention will become apparent to those skilled in the art from the following detailed description of a best mode presently contemplated for practicing the invention by reference to certain preferred methods of the invention, taken in conjunction with the accompanying figures of drawing, in which:

FIG. 1 is a phantom front view of a patient with exemplary locations for harvesting mesenchymal stem cells, and for injecting the harvested stem cells, after their modification by co-culturing with body cells of the type to be mimicked, into the cardiovascular system for introduction at the site of the compromised cells to be repaired or replaced; and

FIG. 2 is a detail view of a location for antegrade injection of the cells.

### DETAILED DESCRIPTION OF THE PRESENTLY CONTEMPLATED BEST MODE OF PRACTICING THE INVENTION

In an exemplary method that applies the principles of the present invention to repair or replacement of myocardial tissue, the injured cardiac tissue is subjected to invasion by stem cells, specifically mesenchymal stem cells that have been modified through co-culturing to mimic the specific natural myocardial cells such as beating cells. These implanted autologous cells undergo subsequent differentiation into beating cardiomyocytes that are mechanically and electrically linked to adjacent healthy host myocardium, thereby resembling newly formed and functionally active myocardium. They serve to correct a problem such as a slow or too rapid heart rate.

Unlike the invention disclosed in the '739 application, where transplantation of autologous adult stem cells derived from the same individual that suffers the infarction is the approach used to repair failing myocardium, the present invention utilizes patient-derived mesenchymal stem cells that have been modified by co-culturing with cells of the type that are to be repaired or replaced. These cells are then injected or implanted into the specific site such as the area of the sinus node in the right atrium for invasion of the injured cardiac tissue, where the stem cells firmly attach and subsequently undergo differentiation into beating cardiomyocytes that are mechanically and electrically linked to adjacent healthy host myocardium. Adhesion of the injected stem cells and their local growth may be confirmed by noninvasive and invasive means such as magnetic resonance tomography (MRT) or intravascular electrical mapping or intravascular ultrasound. For separation of the modified stem cells from the co-cultured animal or fetal pattern-giving cardiomyocytes, markers may be introduced into the stem cells or pattern-giving cells or both before they are separated from each other and injected or transplanted into the myocardial tissue to be repaired.

According to the present invention, mesenchymal stem cells derived from the patient himself or herself are used to repair or replace compromised cells of the heart that have suffered damage or necrosis as a result of an MI attributable to blockage of one or more coronary arteries, or as a result of other heart disease or disorder. The stem cells are harvested from established mesodermal layers, bone marrow, fat or epidermis of the patient. These stem cells are then co-cultured with pattern cells having the very specific function of those that have been compromised in the patient—such as natural pacemaker cells that possess the property of beating spontaneously but, for safety and immunologic considerations, are not deemed to be suitable for injection themselves. The co-culturing may be performed using a Petri dish, or other medium for batch processing. In any event, culturing is continued for a period of several days (e.g., 5 to 20 days). The necessary period of co-culturing is recognized as having been completed when the mesenchymal stem cells are observed to have acquired the very specific features, properties and/or functions of the pattern cells. The mesenchymal are viewed as the most pluripotential stem cells in that they are not fixed as to development, and they have the capacity to affect more than one organ or tissue. Applicant has found that mere contact with pattern cells and exposure to the media and the expression of genes and release of certain proteins, can transform mesenchymal stem cells to mimic the specific functions of the pattern cells.

This means that the patient's own stem cells can be brought together for a certain limited period of time with pattern cells whose properties, features or functions are to be duplicated, and the patient's body cells take on the properties, features or functions of the pattern cells. It remains, then, to separate the two types of cells once again, so as to identify and re-inject into the patient only those cells that were originally harvested from the patient, albeit now modified. Toward that end, the mesenchymal stem cells are marked before the co-culturing is performed, to allow them to be identified later.

One exemplary method of marking is to label the patient's cells (i.e., the mesenchymal stem cells originally derived directly from the patient) with a green fluorescence protein, GFP, with introduction into the stem cell genome by liposomal gene transfer. Then, if the cells taken from the co-culture are found to express GFP in a genome, the patient's autologous cells can be sorted out by first labeling the GFP-expressing cells. Those cells are then processed through a conventional FACS machine to sort and re-sort the cells.

The labeling of these autologous body cells has the added benefit that even if only 90% are labeled, and only that 90% of the desired cells are identified so that 10% are lost, the identified cells are the ones sought, i.e., the correct ones. Whereas, if only the pattern cells to be mimicked were labeled, a harvesting of those cells from the culture would yield a final contamination involving undesirable cells. That is, rather than achieving the desire to obtain 90% of the cells that are correct, some 10% of the wrong cells would be selected. So by the proper labeling of the cells, the selected cells are correct and are then injected at or transplanted to the site where they perform their specific functions in the patient's body.

The present invention is not limited solely to cultivating and injecting pacemaker-like cells at the site of the damaged or dead tissue of the heart, but may also be used to cure other kinds of rhythm disorders that result from a slow conduction in the heart. For example, ischemia or necrosis will result in a zone containing some dead myocardial tissue. And around that zone are some surviving, but slowly conducting cells with an action potential of perhaps something approximating, say, −40 to −60 millivolts. Hence, these cells depolarize slowly and they conduct slowly. This induces reentry phenomena that induce and maintain ventricular tachycardias, a situation that is very common in patients with previous infarctions or damaged myocardium. Cultivation and injection of these new stem cells can correct the problem of slow conduction by implanting cells that have an appropriate fast conduction, thereby eliminating the reentry gap.

The new cells for correction of areas of slow conduction are injected and implanted through the antegrade flow via an artery, and enter the arterioles and the capillaries where they settle and convert into myocardium as their micro-environment conditions them to proper behavior. But a serious problem arises if the artery through which the injection was to take place is occluded and cannot be re-opened to allow re-commencement of antegrade perfusion. This is because the mesenchymal cells that are intended to provide this mimicking behavior will grow and build functional myocardial tissue only if an adequate blood supply exists. The problem is particularly prevalent in older cardiac patients.

According to another aspect of the present invention intended to alleviate or eliminate this problem, the stem cells that are ultimately to be injected for invasion of the predetermined site of the compromised cells are transfected with the growth factor VEGF. By genetic incorporation, the cell can be made to produce increased levels of VEGF. Thus, mesenchymal stem cells that are derived from ordinary fat or bone tissue of the individual patient can be genetically engineered to express an enhanced level of VEGF, which possesses the capability to promote significant growth of new capillaries. But it still remains to re-establish the blood flow to enable this new growth to take place. The applicant herein observed some time ago that irrespective of how long an arterial vessel is occluded, the capillaries and the venous returns along with the arterials are still functioning, sufficiently so that if a contrast dye were injected into the occluded vessel, the dye would be observed to find its way into the venous circulation.

So the venous side of the circulation that is still present in the case of an occluded artery is taken advantage of to enable invasion of the predetermined cell site by retrograde passage. In essence, just as dye through a catheter, or a cell can be injected into the arteries (i.e., into the arterial circulation by antegrade flow), the modified and VEGF-producing mesenchymal cells can be implanted through the right side of the heart. This may be achieved, for example, by delivery of the repair/replacement cells via the lumen of a catheter or via wire insertion, into the coronary sinus and thence into the specific branch of the venous vessels at that location. The delivery may be accessed from the right atrium where the coronary sinus ends, so that the highly specific region of the venous system that parallels the occluded artery may be addressed.

For each artery, a parallel vein can be identified. If an artery is occluded, its parallel vein can be entered for retrograde injection of the modified VEGF-producing cells. At the outset, these cells are only able to survive in a border zone at the site of the compromised cells they are to repair or replace, where some minimum blood flow still exists through collateral vessels to supply oxygen to at least sustain their life but not allow them to actively contract. By virtue of the life-sustaining retrograde injection of the VEGF-producing cells, an adequate number of them can and will engraft themselves in the border zone for growth of new capillaries to aid in reducing the total ischemic area. The border zone is thereby expanded until repair or replacement of the compromised cells is fully or substantially complete following repetitive sessions of injection of cells separated by one to 4 weeks.

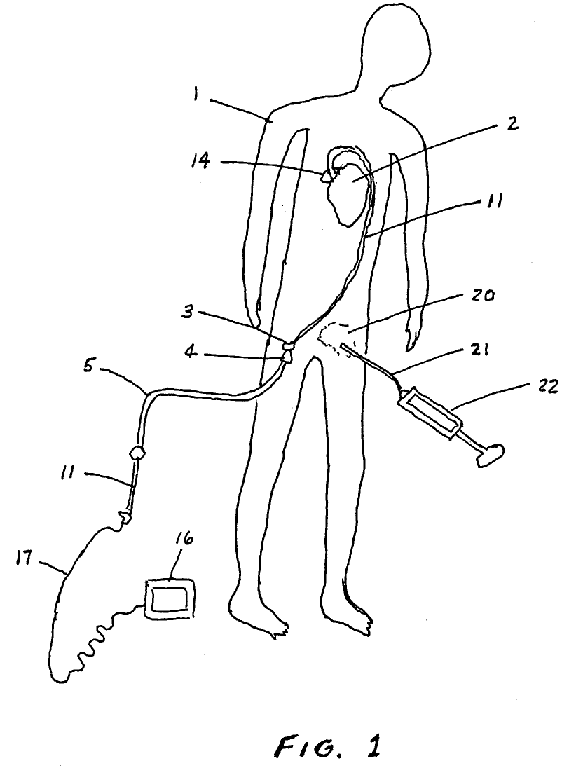

Reference is now made to the accompanying Figures of drawing as an adjunct for describing an exemplary process. It should be noted at the outset that the Figures are not intended to be to scale, nor to do more than serve as a visual aid to the description. The mesenchymal stem cells are harvested in any one of known ways, such as by bone marrow tap or from adipose tissue, for identification, separation and cultivation. In an exemplary technique, and referring to FIG. 1, subcutaneous adipose tissue 20 is obtained from a liposuction procedure on the patient 1 during local anesthesia. In this procedure, a hollow canule or needle 21 is introduced into the subcutaneous space through a small (approximately 1 cm) cut. By attaching gentle suction by a syringe 22 and moving the canule through the adipose compartment, fat tissue is mechanically disrupted and following the solution of normal saline and a vasoconstrictor epinephrine, a lipoaspirate of 300 cc. is recovered (retrieved) within the syringe. The lipoaspirate is processed immediately according to established methods, washed extensively in phosphate buffered saline (PBS) solution and digested with 0.075% collagenase. The enzyme activity is neutralized with Dulbecco's modified eagle medium (DMEM) containing 10% FBS (fetal bovine serum) and, following a centrifugation at 1200 G for 10 minutes, a high density cellular pellet is obtained. Following filtration through an appropriately tight Nylon mesh in order to remove cellular debris, the cells are then incubated overnight in a control medium of DMEM, FBS, containing an antibiotic, antimycotic solution. After the firm attachment of the stem cells to the plate, they are washed extensively with PBS solution to remove residual non-adherent stem cells other than mesenchymal. Red blood cells are lysided by water and ammonium chloride. Further cellular separation may be conducted by separation with monoclonal antibodies coated on magnetic beads or by FACS.

The thusly-derived mesenchymal stem cells are next labeled by marking them with GFP or any other surface marker or genetic marker before any culturing is performed, so as to enable these cells to be identified afterward. Toward that end, the GFP is introduced into the stem cell genome by liposomal or viral gene transfer. Cells may then be identified after the co-culturing step by fluorescence microscopy. As part of the procedure, stem cells may also be marked by other cell specific markers.

Before co-culturing the marked stem cells with the cells whose highly specific features, properties or functions are to be mimicked, e.g., natural pacemaker pattern cells that have been harvested from embryonic or neonatal animals and themselves identified and separated by labeling and sorting steps such as by surface marker connexin 40 and/or 43 labeling, the stem cells can be subjected to transfection with other factors such as VEGF growth factor. This is performed by genetic incorporation through a viral or lipsomic promoter, to increase the levels of production of VEGF by the cells. As a result, the mesenchymal stem cells that are derived from the normal fat, blood or bone marrow tissue of the patient are genetically engineered to express an enhanced level of VEGF, so as to promote significant growth of new capillaries when the cells are injected, especially if it is found that the patient's occluded coronary artery cannot be re-opened, and resort must be made to retrograde flow through the patient's venous circulation at the infarction site to be invaded with the modified stem cells.

Next, the co-culturing of the cells is performed. The mesenchymal stem cells derived from the patient and the beating (e.g., embryonic depolarizing) pattern heart cells are cultured together. This may be performed in a Petri dish for a period of nominally about 10 to 14 days, after which applicant herein has found that mere contact between the two cell types and exposure to the media and the expression of genes and release of certain proteins, the mesenchymal stem cells are transformed into the same cell type as the pattern cells, for example beating heart cells. The microenvironment created mimics the conditions in the body to guide the autologous pluripotential cells regarding which developmental direction to take. The period required to achieve this transformation may be shorter or longer, and can be determined more precisely by monitoring the status of the stem cells, whose marker enables them to be readily identified. If desired, a larger batch nrocessing technique may be used. In any event, the previous marking of the cells allows them to be identified for separation from the pattern cells once again. These labeled and separated autologous cells are then ready for implantation in the patient at the site of the tissue to be repaired or replaced, as cells that originally came from the patient and hence unlikely to create problems of rejection or late malignancy.

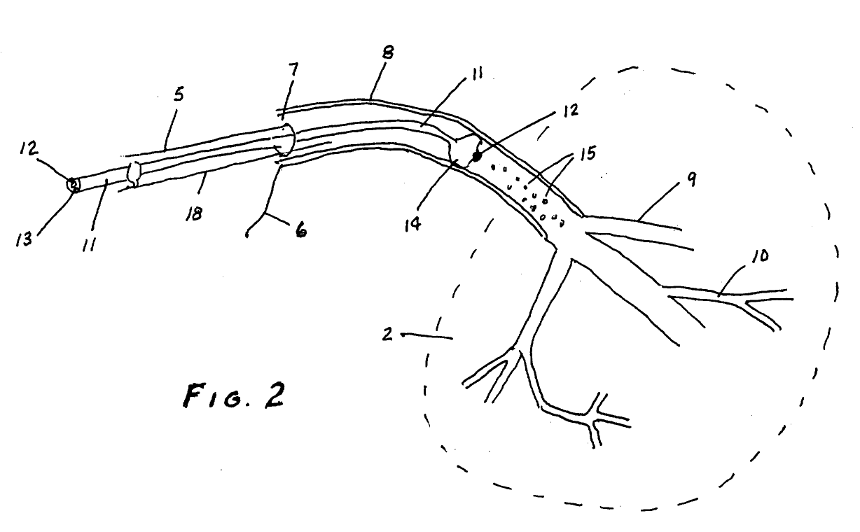

Referring to FIG. 2 as well as to FIG. 1, the modified mesenchymal stem cells with or without enhanced VEGF-production are transplanted in the donor patient by intracoronary, transcoronary retrograde through the coronary sinus application for myocardial repair or replacement of the compromised cells, the latter being either damaged or necrose at the site of the injury and damage. Another technique to apply the cells is to inject them locally into the damaged tissue such as at an anatomic site in the right atrium or at a slow conduction zone in the ventricle. In the absence of having encountered one or more occluded coronary arteries that caused the infarction and which cannot be re-opened, the process of cell injection into the antegrade circulation is performed by first introducing a balloon catheter 11 into the cardiovascular system at the patient's groin 3 using an introducer 4, and through a guiding catheter 5 over a guide wire 18 into the aorta 6 and the orifice 7 of a coronary artery 8 of the heart 2 at or in the vicinity of the designated site. The failed tissue is supplied with blood through artery 8 and its distal branches 9 and 10. The cells are then injected through the inner (central) lumen 12 of the balloon catheter 11 by means of an injection syringe 16 and connecting catheter 17 to an entry point of the central lumen at the proximal end of catheter 11. The exit point of the central lumen 12 is at the distal end of catheter 11 which has been advanced into the coronary artery 8 in proximity to the site of the desired repair. The cells 15 are thereby delivered to this site by means of slow infusion over an interval of from 25 to 30 minutes, for example.

A problem encountered in attempting to do this resides in the fact that normally anything inside the blood vessel, including these cells, is separated from the parenchymatous organ or the tissue outside the vessel. In principle, blood flows through the larger arteries into the smaller arteries, into the arterials, into the capillaries, and then into the venous system back into the systemic circulation. Normally, the cells would be prevented from contacting the tissue to be repaired because of the endothelial lining and layer of the vessel that protects the tissue. However, under certain circumstances this barrier is overcome, and the cells can attach to the inside of the vessel, migrate and proliferate in the adjacent tissue. These circumstances are facilitated in the case of an acute myocardial infarction, and the increased pressure in the injection system promotes the injected cells to overcome the barrier.

The endothelial ischemic damage owing to the infarction allows white blood cells, especially granulocytes and macrophages, to attach via integrins to the endothelial layer. The endothelial layer itself is dissolved in places by the release of hydrogen peroxide (H₂O₂), which originates from the granulocytes. This mechanism produces gaps in the endothelial layer that allow the stem cells to dock to the endothelial integrins and also to migrate through these gaps into the tissue to be repaired. An adjacent factor that enables the stem cells to migrate into the organ tissue is referred to as a stem cell factor that acts as a chemo-attractant to the cells.

One is then still faced with the problems of allowing enough of the repair cells to migrate into contact with the failing tissue and of achieving a high number of transplanted cells in the tissue. This is the principal reason for using a balloon catheter 11 or some other mechanism that will allow the physician (operator) to selectively block the antegrade blood flow and the retrograde stem cell flow. A balloon catheter is preferred because it is a well known, often used and reliable device for introduction to a predetermined site in a vessel such as a coronary artery, to be used for angioplasty for example. In the process of the invention, the balloon 14 of catheter 11 is inflated with biocompatible fluid through a separate lumen 13 of catheter 11 to occlude coronary artery 8 and its distal branches 9 and 10, thereby causing perfusion through the vessel to cease. Inflation of the balloon may be commenced immediately before or at the time of injection of the stem cells through the inner lumen of the catheter, and is maintained throughout the period of injection. This enables the desired large number of adhesions of the cells 15 to the failing tissue to be achieved, because the absence of blood flow at the critical site of this tissue to be repaired has several advantageous effects. It prevents what would otherwise result in a retrograde loss of injected cells, an inability to increase the pressure at the injection site to overcome the endothelial barrier and to force the cells through the gap, and an antegrade dilution with blood flow of the cells being injected to that location through the catheter 11.

The blockage is maintained for a relatively short period of time, preferably on the order of fifteen minutes, and in any event sufficient to allow a high concentration and considerable number of cell attachments to the tissue at the designated site, that will tend to guarantee a successful repair. This repair will extend as well to any failing tissue that may result from the blockage itself. In the case of a slow infusion of the cells, the period of blockage is maintained longer by steady inflation of the balloon over the injection period, say, up to about 30 minutes, for enhancement of contact and adherence of abmSC-P to the endothelium. The balloon is deflated, and the balloon catheter is removed from the patient after the procedure.

Previously reported studies have invariably employed a surgical approach for the application of the cells to be transplanted in a large myocardial infarction area. Even if 5 to 10 sites of injection are performed with small needles, the complete inner, medial and outer layers of the myocardium are never covered if the aim is to transplant cells to a larger area of myocardial failure, as would be the case with a myocardial infarction. However, this technique is sufficient if only a small number of cells such as pacemaker cells are to be transplanted.

If, however, the aim is to rebuild a very specific failing myocardial function such as the lack of generation of a pulse with the consequence of a too slow heart rate and the need for an artificial pacemaker, then the transplantation of specifically modified pacemaker cells at the site of the normal sinus node location at the junction of the superior vena cava and the right atrium locally is sufficient. For this purpose, the injection of the specific cells by a needle catheter at the specific site is one option in addition to a catheter based application of the modified mesenchymal stem cells in a carrier such as collagen to increase their mechanical stability and transplantation efficiency.

A process for injection of the modified and VEGF-producing mesenchymal stem cells into the retrograde circulation may be used in a situation where interventional cardiology is unable to successfully restore blood flow following an acute MI after an occlusion of a coronary artery. In that case, the cells are preferably injected through a catheter advanced through the right atrium into the coronary sinus for entry into the specific branch of the venous vessels, and thereby the retrograde circulation, for invasion of the compromised tissue at the infarction site.

The invention is not limited to cellular repair of damaged myocardial tissue. Rather, the process by which the repair is performed may be applied to the brain in the case of a patient having suffered a cerebral infarction, to the eye, the ear, the kidney, peripheral arteries and perfusion in the leg, or to the liver and pancreas. Previous studies have indicated that stem cells have the capacity to replace neural cells in the brain and therefore overturn the consequences of an acute vascular stroke. In this case, the injection catheter is advanced to the site of the damaged tissue through an appropriate arterial path into the applicable region of the patient's brain. Blockage of blood flow in this case would add a period (e.g., 5 minutes) of limited blood supply but would enable the cells to overcome the endothelial barrier.

Other possible body organs having damaged tissue to be repaired by the process of the invention include the pancreas, the liver, and the kidneys. The pancreas has a duct through which pancreatic enzymes are delivered into the intestines, and which can be accessed in a retrograde manner by endoscopic retrograde choledocho-pancreaticography (ERCP). Failing tissue in the case of a diabetic patient means that the pancreatic cells therein no longer produce sufficient insulin for the patient's needs. By means of a small fiberglass instrument a small balloon catheter may be introduced into this duct, and the balloon inflated to occlude the duct during delivery of stem cells through the catheter's inner lumen to the site of the damaged tissue, so as to prevent the injected cells from being washed out into the intestines and thereby enhance large scale adhesions and penetration of the cells to the target tissue.

An analogous procedure is used for repair of damaged tissue of the liver, through the bile duct system. Here also, it is important to overcome the barrier of the normal bile duct with pressure that can be generated only if the balloon is inflated while the cells are slowly injected. The pressure distally of the injection site increases as more and more cells are injected. Repair of failing tissue in the kidney(s) may be repaired by an analogous procedure in the case of a renal infarction. Also, the bone marrow might be replaced by this microenvironment reading pattern method using autologous pluripotential non marrow-derived stem cells.

Some of the processes noted above are described in greater detail below.

Culturing to extract cells from fat tissue may be performed, for example, in the following manner. For patients with damaged organs, such as the heart following an MI as an example, an excision or liposuction of fat tissue is performed to obtain an autologous cell pool. The cells are then isolated from the tissue by two steps. The first is mechanical disruption of the tissue, which yields smaller pieces of tissue into which enzymes can penetrate more easily. The second of these steps is enzymatic digestion of the smaller pieces (e.g., with Collagenase 0.075% and 2.5 mM CaCl in Phosphate Buffered Saline (PBS) for 1 to 3 hours at 37° C. on a shaker). After the digestion the Collagenase is inactivated by the addition of 5 mM EDTA (ethylenediaminetetraacetic acid). The cell suspension is filtered through a 100 μm mesh to separate single cells from residual connective tissue pieces and centrifuged at 450 G for 10 minutes. The supernatant is discarded and the cell pellet is re-suspended in a sufficient amount of growth media (e.g., 20 ml growth media (alpha-Modification of Eagle's Medium with Penicillin/Streptomycin and L-Glutamine and 20% fetal bovine serum) for a 175 cm² tissue culture dish). Cells are then grown in a tissue culture incubator at standard conditions (37° C., saturated humidity, 5 volume % of CO₂ atmosphere).

The cell suspension will settle in the culture dish over the next several hours and a fraction of the cells will start to adhere to the dish surface. Non-adhering cells are washed away during subsequent media changes and standard sub-cultivation steps: 1) washing a 175 cm² tissue culture dish with 10 ml of HANKS balanced salt solution; 2) incubation for 5 minutes with 2 ml Trypsin 0.25%/EDTA 0.1% to detach cells; 3) inhibition of Trypsin by addition of equal amounts of growth media.

The following steps are based on the observation that to differentiate the fat tissue derived cells (FTDC) from the cells of interest to aid in repair of the damaged organ, a direct cell-cell contact between the two cell types is necessary. One technique to achieve the differentiation of the FTDC into cardiomyocytes is a co-cultivation with neonatal rat cardiomyocytes. An integral part of this process is to assure the co-cultured cells can be separated again afterwards. This separation can be based on: a) surface antigens, because two different species are used, or b) fluorescent labeling of one cell type, and separation based on the labeling.

An example of the labeling/separation techniques includes fluorescent membrane labeling, in which the plastic adherent cells are transiently labeled with a fluorescent membrane marker such as PKH26 (Sigma Aldrich) or Dil-acetylated LDL (Cell Systems). These markers are integrated into the cell membrane, and the fluorescent signal can be followed for a prolonged period of time. A diffusion of the marker substance into unlabeled cells in culture is thought to be non-existing.

For the co-cultivation aspect, after neonatal cardiomyocytes have been obtained by standard extraction methods as outlined above, they are plated on tissue culture treated surfaces with a density range of 10,000 to 30,000 cells per cm² and grown for several hours. Following appropriate labeling of the FTDC with one of the membrane markers (following manufacturer's protocols) the cells are added to the neonatal cardiomyocytes in a 1:1 ratio. Standard growth media previously discussed herein may be used. Suitable transcription factor-inducing substances such as 5-azacytidine or suitable growth factors such as VEGF or FGF (fibroblast growth factor) can be added in general to the tissue culture media to facilitate the differentiation process.

The co-cultivation tissue culture is monitored by microscopy, and the staining of small samples for lineage specific markers by immunohistochemistry. After a sufficient time period of co-cultivation, ranging from several hours to several days, the cells are treated with trypsin as described above to obtain a single cell solution. In addition, the cells are centrifuged at 450 G for 5 minutes and re-suspended in PBS to perform fluorescence activated cell sorting (FACS) for separation.

In the latter step, the FTDC that were labeled with a fluorescent marker can be separated by means of FACS, in which the single cell suspension in PBS may be used. FACS involves the scanning of cells that are lined up in a capillary stream one after the other to pass a laser beam that excites the fluorescent dye (using a wavelength specific for the used dye). The excitation causes light to be emitted on a different wavelength (itself specific for the dye), for detection. With this technique, every cell is scanned for the marker and marker positive and negative cells are subsequently sorted into different vials. Greater than 95%-pure populations of cells carrying the marker are obtained, and repetitive processing allows the effectiveness to approach 100%.

Another means to label cells with fluorescent markers that can be used for sorting afterwards is fluorescent labeling of cells by liposomal transfection—transient transfection with plasmids encoding fluorescent proteins (e.g., GFP). The plasmids may be introduced into FTDC by standard liposomal transfection as outlined above. The FTDC will express this genetic information encoded on the plasmid and produce GFP transiently, and the GFP can be detected and sorted by FACS.

Yet another technique for cell labeling is fluorescent labeling of cells by viral gene transfer. Here, adenoviruses or adeno-associated (AAV) viruses may be used to introduce fluorescent gene markers into cells. Thereafter, FACS is used for sorting the cells.

Also, FACS may be used to separate the co-cultured cells by surface antigens. For example, if the FTDC are derived from humans and the cardiomyocytes are derived from animals, the cells can be distinguished by their expression of different cell surface antigens. The most important class is called major-histocompatibility antigens (MHC antigens) or in humans: human-leucocyte associated antigens (HLA antigens). Proteins (in this context called antibodies (AB)) that are conjugated (coupled) with a fluorescent marker and that recognize and bind to these HLA antibodies are commercially available. One suitable antibody against the HLA-DR antigen is available conjugated to fluorescent dyes such as phycoerythrin or FIT-C.

After obtaining a single cell suspension in PBS of the co-cultured cells as described above, the concentration of cells should be about 1×10⁶ cells in 50 μl of PBS in a suitable vial. The fluorescence-conjugated antibody (conj-AB) is added (as recommended in the manufacturer's description, normally about 5 μl of conj-AB) and incubated on ice in the dark for 15-30 minutes. Thereafter, washing of residual AB is performed by adding 2 ml of PBS, and the cells are centrifuged in a blood bank centrifuge at 750 G for 45 seconds. The supernatant is discarded and the cells are re-suspended in 2 ml PBS and centrifuged at 750 G for 45 seconds. The cells are again re-suspended, this time in 1 ml PBS and then sorted by FACS scan. The AB binds to the HLA-DR antigen, which is only present on the human FTDC, and the human cells will be sorted as positive fluorescent. The animal cells will not bind the AB and will be sorted as negative fluorescent. After repeated sorting, the recovery of pure human FTDC population approaches 100%.

Separation of co-cultured cells may also be achieved by surface antigens through magnetic beads. This methodology for separation of the co-culture is similarly based on the fact that the FTDC are derived from humans and the cardiomyocytes are derived from animals, and that the cells can be distinguished by their expression of different cell surface antigens. The anti-HLA-DR antibody can be used here, as well. This AB will be coupled to magnetic beads, and they are incubated together with the cells in single cell suspension. The human cells, which express the HLA-DR antigen on their surface, bind to the AB coupled to the bead. All unbound cells (the animal cardiomyocytes) are washed off in subsequent steps, and the bead-bound cells can be recovered in a final step by elution.

As used herein, the term non body cells refers to cells that are not derived from the patient himself or herself, and include (1) human embryonic or neonatal cells, (2) animal embryonic or neonatal cells, (3) adult stem cells, and (4) artificial cellimes. The non body cells may be transfected with specific genetic markers initially, so as to acquire the features of the failing tissue to be repaired by a process of the invention.

Also as used in the specification, the term microenvironment refers to an environment suitable to sustain and nurture live cells. For example, the portion of the myocardial tissue affected by an MI includes a nucleus of dead tissue and a border zone of at least some live tissue. Pluripotential cells implanted in the nucleus will not repair or replace the dead tissue because of the absence of a microenvironment. To do so, cells present in the region of the implantation should have both surface markers and the capability to produce protein.

Although a presently contemplated best mode of practicing the invention has been disclosed by reference to certain preferred methods, those skilled in the art will recognize from a consideration of the foregoing description that variations and modifications may be made without departing from the spirit and scope of the invention.

What is claimed is:

1. A method of cellular repair or replacment of failing tissue of an organ in a patient's body, which comprises the steps of:
co-culturing pluripotential cells harvested from the patient's body with other cells acquired from a source other than the patient's body (non body cells) and having specific functions of the failing tissue to be mimicked by the pluripotential cells, for a period of time sufficient for the pluripotential cells to be modified by having acquired said specific functions of the non-body cells; and
separating the body and non-body cells and applying only the body cells to the site of the failing tissue to be repaired or replaced.

2. The method of claim 1, wherein the organ is the patient's heart, the failing tissue is a region of myocardial tissue, and the step of applying to the site includes injecting the modified pluripotential cells to the site of failing tissue.

3. The method of claim 2, wherein the step of injecting includes delivering the modified pluripotential cells through a coronary artery for antegrade circulation to a location of a myocardial injury constituting said site.

4. The method of claim 2, wherein the step of injecting includes delivering the modified pluripotential cells into the patient's venous system for retrograde circulation to a location of a myocardial injury constituting said site.

5. The method of claim 1, including marking the pluripotential cells prior to the co-culturing step to enable them to be segregated from the non-body cells after having acquired said specific functions thereof.

6. The method of claim 1, including transfecting the harvested pluripotential cells with vascular endothelial growth factor (VEGF) to promote growth of new capillaries when said pluripotential cells are injected to invade the site of the failing tissue.

7. The method of claim 4, wherein the step of injecting is performed after determining that an occluded vessel responsible for the myocardial injury is not amenable to being re-opened.

8. The method of claim 7, including transfecting the harvested pluripotential cells with VEGF growth factor to promote growth of new capillaries when said modified pluripotential cells are injected to invade the site of the failing tissue, so as to maintain blood flow to at least a border zone of living tissue at said site.

9. The method of claim 1, wherein said pluripotential cells are derived from the patient's adipose tissue.

10. The method of claim 1, wherein said pluripotential cells are derived from the patient's blood.

11. The method of claim 1, wherein said pluripotential cells are derived from the patient's bone marrow.

12. The method of claim 1, wherein said period of time is in a range from about 5 to about 20 days.

13. A method of repairing tissue of an organ in a patient's body, comprising co-culturing pluripotential cells obtained from the patient's own body with non body cells obtained from another source having a specific function of the tissue to be repaired for mimicking by the pluripotential cells, maintaining the culture for a period of time sufficient for modification of the pluripotential cells by acquisition of said specific function of the non body cells; and implanting the modified pluripotential cells at the site of the tissue to be repaired.

14. The method of claim 13, wherein the specific function of the tissue to be repaired is obtained in the non body cells by a selection according to specific cell markers.

15. The method of claim 13, wherein the specific function of the tissue to be repaired is obtained in the non body cells by genetic modification of the non body cells.

16. The method of claim 13, wherein the specific function of the tissue to be repaired is obtained in the non body cells by a selection according to specific cell markers and by genetic modification of the non body cells.

17. The method of claim 13, including segregating the modified pluripotential cells from the cells co-cultured therewith before performing the implanting step.

18. The method of claim 17, including marking the pluripotential cells before performing the culturing step, for distinction from the non body cells during the segregating step.

19. The method of claim 13, wherein the step of implanting includes applying the modified pluripotential cells locally at a specific site of tissue to be repaired.

20. The method of claim 13, wherein the organ is the patient's heart, the tissue to be repaired is myocardial tissue, and the step of implanting includes injecting the modified pluripotential cells into a vessel leading to said site.

21. The method of claim 20, wherein the step of injecting includes delivering the modified pluripotential stem cells through a coronary artery for antegrade circulation to a location of a myocardial infarction constituting said site.

22. The method of claim 20, wherein the step of injecting includes delivering the modified pluripotential stem cells into the patient's venous system for retrograde circulation to a location of a myocardial infarction constituting said site.

23. The method of claim 22, wherein the step of injecting is performed after determining that an occluded vessel responsible for the infarction is not amenable to being re-opened.

24. The method of claim 22, including transfecting the harvested mesenchymal stem cells with VEGF growth factor to promote growth of new capillaries when said stem cells are injected to invade the site of the tissue to be repaired, so as to maintain blood flow to at least a border zone of living tissue at said site.

25. A method of repairing or replacing failing tissue of an organ in a patient's body, comprising harvesting mesenchymal stem cells from the patient's body, transfecting the harvested mesenchymal stem cells with VEGF growth factor, and applying the VEGF-transfected mesenchymal stem cells to a site of failing tissue to be repaired or replaced.

26. The method of claim 25, wherein the step of applying comprises applying the VEGF-transfected mesenchymal cells locally into the failing tissue directly at said site.

27. The method of claim 25, wherein the organ is the patient's heart, the failing tissue is myocardial tissue, and the step of applying comprises injecting the VEGF-transfected mesenchymal stem cells into a blood vessel leading to said site of failing tissue.

28. The method of claim 27, wherein the step of injecting comprises delivering the VEGF-transfected mesenchymal stem cells through a coronary artery for antegrade circulation to said site of failing tissue.

29. The method of claim 27, wherein the step of injecting comprises delivering the VEGF-transfected mesenchymal stem cells into the patient's venous system for retrograde circulation to said site of failing tissue.

30. The method of claim 25, further comprising co-culturing said VEGF-transfected mesenchymal stem cells with natural body cells obtained from a source other than the patient's body having specific functions of the failing tissue for mimicking by the mesenchymal stem cells, maintaining the co-culture for a period of time sufficient for modification of the mesenchymal stem cells by acquisition of said specific functions of the natural body cells, and applying the modified mesenchymal stem cells to the site of the failing tissue.

31. A method of treating cardiac arrhythmias that originate from an area of slow impulse conduction in the heart, comprising harvesting pluripotential stem cells from the patient's body, and applying the harvested pluripotential stem cells to said area to increase the slow impulse conduction toward normal conduction and thereby avoid local reentry phenomena.

32. The method of claim 31, wherein the step of applying comprises injecting the harvested pluripotential stem cells locally and directly to said area with a needle catheter.

33. The method of claim 31, wherein the step of applying comprises delivering the harvested pluripotential stem cells locally to said area by catheter.

34. The method of claim 31, wherein the step of applying comprises delivering the harvested pluripotential stem cells to said area by antegrade circulation.

35. The method of claim 31, wherein the step of applying comprises delivering the harvested pluripotential stem cells to said area by retrograde circulation.

36. The method of claim 31, further comprising co-culturing the harvested pluripotential stem cells with cells obtained from a source other than the patient's body (non body cells) having specific functions of the failing tissue for mimicking by the pluripotential stem cells, and maintaining the co-culture for a period of time sufficient for modification of the pluripotential stem cells by acquisition of said specific functions of the non body cells, before said step of applying.

37. The method of claim 31, further comprising transfecting the non body cells with specific genetic markers so as to acquire the features of the failing tissue to be repaired, before said applying step.
